(19) United States
(12) Patent Application Publication
(10) Pub. No.: US 2005/0239837 A1
(43) Pub. Date: Oct. 27, 2005

(54) Process for production of highly pure donepezil hydrochloride

(76) Inventors: Arie L. Gutman, Haifa (IL); Marina Etinger, Nesher (IL); Boris Tishin, Haifa (IL); Boris Pertsikov, Nesher (IL); Boris Fedotev, Haifa (IL); Alexander Vilensky, Haifa (IL); Pavel Potyabin, Haifa (IL); Gennady A. Nisnevich, Haifa (IL)

Correspondence Address: STERNE, KESSLER, GOLDSTEIN & FOX PLLC, 1100 NEW YORK AVENUE, N.W., WASHINGTON, DC, 20005, US

(21) Appl. No.: 11/112,002

(22) Filed: Apr. 22, 2005

Related U.S. Application Data
(63) Continuation-in-part of application No. 10/640,419, filed on Aug. 14, 2003.
(60) Provisional application No. 60/444,653, filed on Feb. 4, 2003.

(30) Foreign Application Priority Data
Aug. 14, 2002 (IL) 151253

(51) Int. Cl. A61K 31/445; C07D 211/06

(52) U.S. Cl. 514/319; 546/206

(57) ABSTRACT

Disclosed is a process for production of highly pure donepezil hydrochloride in the form of polymorph I that does not involve the isolation of donepezil base. The disclosed process involves intramolecular cyclization of N-benzyl-2-(3,4-dimethoxybenzyl)-3-(4-piperidine)propionic acid followed by treatment with HCl.

### BACKGROUND OF THE INVENTION

1. Field of the Invention

The present invention relates to a process for the preparation of highly pure donepezil hydrochloride in the form of polymorph I.

2. Related Art

5,6-Dimethoxy-2-(1-benzyl-4-piperidinylmethyl)-1-indanone, known under the international nonproprietary name (INN) donepezil, is an acetylcholinesterase inhibitor (The Merck Index, 13th Ed., 2001, Monograph #3453).

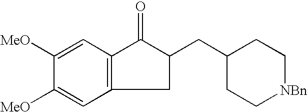

Tablets containing the hydrochloride salt of donepezil are marketed in the United States of America under the proprietary name ARICEPT (Eisai). This product is orally administrated and clinically used as a therapeutic and ameliorating agent for Alzheimer's-type senile dementia, particularly as a prophylactic treatment.

Donepezil hydrochloride in the forms of polymorphs II, IV and V is claimed in U.S. Pat. No. 6,140,321. Donepezil hydrochloride form III is claimed in U.S. Pat. No. 5,985,864. Donepezil hydrochloride in the form of polymorph I was described in the same patents and earlier in Example 4 of U.S. Pat. No. 4,895,841. Donepezil hydrochloride in the form of polymorph I is substantially stable under storage conditions for 1 month at 60° C. (FIG. 18 of U.S. Pat. No. 6,140,321). Polymorph I preserved water content in a narrow range of 4.5 to 5.9% when it was stored at 25° C. under an atmosphere with a relative humidity of 10 to 96% (see FIG. 20 of U.S. Pat. No. 6,140,321). The crystalline donepezil hydrochloride form I is characterized by a x-ray power diffraction spectrum having peaks expressed in degrees 2θ at 9.94, 10.60, 12.66, 13.12, 13.66, 13.86, 14.92, 15.26, 16.08, 16.86, 17.50, 17.58, 18.42, 19.28, 19.80, 19.94, 21.22, 22.00, 22.54, 22.98, 23.60, 23.78, 23.92, 26.46, 28.02 and 29.50 (Claim 12 of U.S. Pat. No. 6,140,321). FIGS. 1 and 21 of U.S. Pat. No. 5,985,864 show a x-ray powder diffraction spectrum of crystalline donepezil hydrochloride form I.

Donepezil hydrochloride is produced by first producing donepezil, which is a free base, and then converting it into a hydrochloride. In other words, donepezil base is used as a precursor for the production of donepezil hydrochloride.

A synthetic route to donepezil is shown in Scheme 1 and comprises the condensation of 5,6-dimethoxy-1-indanone with N-benzyl-4-piperidinecarboxaldehyde followed by reduction of the obtained N-benzyl-5,6-dimethoxy-2-(4-piperidinylmethylene)-1-indanone [III] and column chromatography of the crude donepezil base on silica gel. Donepezil hydrochloride is prepared from the resulting purified donepezil base by treating the base with hydrogen chloride and re-crystallizing the resulting solid from MeOH/i-Pr₂O (Sugimoto et al., J. Med. Chem. 38:481 (1995); U.S. Pat. No. 4,895,841).

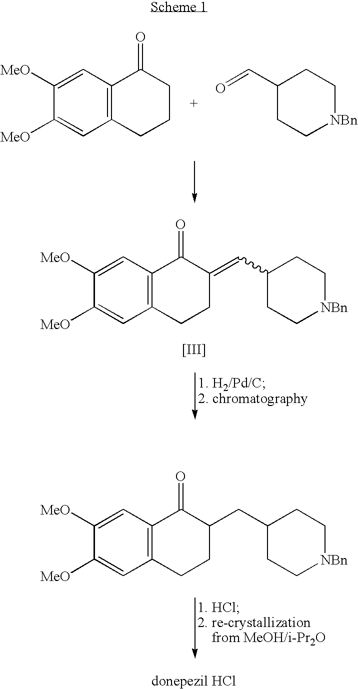

Recently it was proposed to use crystallization for purification of crude donepezil base obtained after the reduction of compound [III] (U.S. Pat. No. 6,245,911).

An alternative process disclosed in WO 00/09483 for preparing donepezil is shown in Scheme 2 and comprises the steps of (a) carrying out the intramolecular cyclization of N-benzyl-2-(3,4-dimethoxybenzyl)-3-(4-piperidine)propionic acid to yield donepezil base and (b) isolating the resulting donepezil base by chromatography or crystallization.

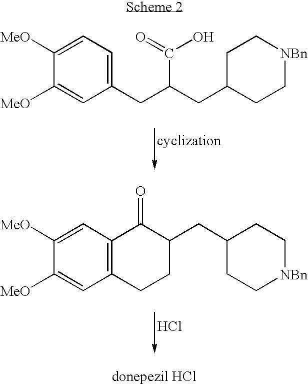

An additional process for production of donepezil hydrochloride involves reacting a donepezil intermediate with halogenated benzyl to obtain a donepezil quaternary ammonium salt, hydrogenation of the quaternary ammonium salt to produce donepezil base, followed by addition of hydrogen chloride to produce donepezil hydrochloride (U.S. Pat. No. 6,252,081).

It should be noted that all of the known procedures for preparing donepezil hydrochloride form I involve the isolation and handling of the intermediate donepezil base prior to converting it into donepezil hydrochloride.

There are several disadvantages in such two-step processes. These include increased cycle time due to increased handling requirements, increased air emissions if drying of the donepezil base is required, and increased total volume of solvent needed. Further, in the prior art, the desired compound is purified by column chromatography, which requires a large amount of solvent and the column is discarded after use, and is thus very disadvantageous in production cost. In addition, a large amount of used solvent is evaporated into the atmosphere. Therefore, column chromatography is preferably not used from the viewpoint of working environment and environmental protection.

### SUMMARY OF THE INVENTION

In view of the foregoing, there is a need in the art to provide a scalable industrial process for synthesizing highly pure donepezil hydrochloride form I hydrate, without the need for isolation, chromatography and re-crystallization of the intermediate donepezil base. It is a further need in the art to provide a process for synthesizing donepezil hydrochloride form I hydrate having a liquid chromatography (LC) purity (by area under a LC peak) of more than 99% and a content of each individual impurity not exceeding 0.02% (by area).

Thus, the present invention provides a process for preparing crystalline donepezil hydrochloride form I hydrate, wherein the obtained salt is characterized by 4 to 6% of water content, a LC purity (by area) of more than 99% and a content of each individual impurity not exceeding 0.02% (by area), which process comprises:
carrying out an intramolecular cyclization of N-benzyl-2-(3,4-dimethoxybenzyl)-3-(4-piperidine)propionic acid or its salt to form donepezil base;
treating the donepezil base with HCl without isolating the donepezil base to form donepezil hydrochloride;
crystallizing the donepezil hydrochloride to give crystalline form I of donepezil hydrochloride; and
maturing the donepezil hydrochloride form I in a damp atmosphere to give donepezil hydrochloride hydrate form I of desired LC purity.

### BRIEF DESCRIPTION OF THE DRAWINGS

FIG. 1 shows a characteristic x-ray powder diffraction pattern of donepezil hydrochloride form I hydrate, according to embodiments of the present invention. Vertical axis: intensity (CPS); Horizontal axis: 2θ (degrees).

FIG. 2 shows the infrared spectrum (diffuse reflectance, DRIFTS) of donepezil hydrochloride form I hydrate in potassium bromide, according to embodiments of the present invention.

FIG. 3 shows the differential scanning calorimetry (DSC) curve of donepezil hydrochloride form I hydrate, according to embodiments of the present invention.

FIG. 4 shows the thermograviometric (TGA) curve of donepezil hydrochloride form I hydrate, according to embodiments of the present invention.

### DETAILED DESCRIPTION OF THE INVENTION

The described invention provides a process for the preparation of highly pure donepezil hydrochloride in the form of polymorph I by cyclization of N-benzyl-2-(3,4-dimethoxybenzyl)-3-(4-piperidine)propionic acid or a salt thereof.

Preferably, the N-benzyl-2-(3,4-dimethoxybenzyl)-3-(4-piperidine)propionic acid or its salt has a LC purity (by area) of at least 97%.

More preferably, the N-benzyl-2-(3,4-dimethoxybenzyl)-3-(4-piperidine)propionic acid or its salt has a LC purity (by area) of at least 98%.

More preferably, the N-benzyl-2-(3,4-dimethoxybenzyl)-3-(4-piperidine)propionic acid or its salt has a LC purity (by area) of at least 99%.

Most preferably, the N-benzyl-2-(3,4-dimethoxybenzyl)-3-(4-piperidine)propionic acid or its salt has a LC purity (by area) of at least 99.9%.

Preferably, cyclization of N-benzyl-2-(3,4-dimethoxybenzyl)-3-(4-piperidine)propionic acid is carried out under Friedel-Crafts reaction conditions, optionally with previous derivatization of the carboxylic group to a halocarbonyl group.

Preferably, the cyclization in the process of the present invention is carried out in the presence of protic acids or Lewis acids or a mixture of protic and Lewis acids. Examples of protic acids are trifluoromethanesulfonic acid, methanesulfonic acid, polyphosphoric acid, fluorosulfonic acid, chlorosulfonic acid, sulfuric acid, hydrogen fluoride, and hydrogen chloride. Examples of Lewis acids are zinc chloride, zinc bromide, aluminum chloride, aluminum bromide, titanium chloride, boron fluoride, phosphorus pentoxide, phosphorus oxychloride, phosphorus pentachloride, phosphorus trichloride, thionyl chloride, and sulfuryl chloride.

The cyclization may be carried out in the presence of a solvent. Preferably, the solvent is a halogenated solvent selected from dichloromethane, chloroform, dichloroethane, tetrachloroethane, chlorobenzene, dichlorobenzene, or may be nitromethane, nitroethane, nitrobenzene, ether and/or mixtures thereof.

After mixing N-benzyl-2-(3,4-dimethoxybenzyl)-3-(4-piperidine)propionic acid with a solvent and a protic acid and/or Lewis acid, the mixture is stirred at a temperature in the range of 0-100° C., preferably in the range of 10-40° C., most preferably in the range of 15-30° C. The cyclization process can be carried out for a time period of 1-10 hours, preferably 2-6 hours, more preferably 3-4 hours.

The carboxylic group of N-benzyl-2-(3,4-dimethoxybenzyl)-3-(4-piperidine)propionic acid can be derivatized to a halocarbonyl group using compounds such as phosphorus trichloride, thionyl chloride or oxalyl chloride (Smith et al., March's Advanced Organic Chemistry, 5th Ed., Wiley, & Sons, Inc., New York, p. 523). The derivatization can be carried out prior to the cyclization process and the derivatized compound added to the cyclization reaction mix. Alternatively, the derivatization can be carried out in situ during the cyclization process.

After the cyclization process the reaction mix can be cooled by pouring the mix into ice. After cooling, the mix can optionally be basified with alkali to a pH of about 9-10. For example the alkali is potassium hydroxide, sodium hydroxide or sodium carbonate. The organic layer is removed, dried over a dehydrating material, e.g., sodium sulfate, and filtered to give a solution of crude donepezil base. Optionally, the aqueous layer can be extracted with solvent, e.g., dichloromethane, and the organic extracts combined prior to drying and filtering. The crude donepezil base with 85 to 99% purity by HPLC may be isolated from the solution and purified by chromatography or crystallization methods. But it is preferable to treat the crude donepezil base with hydrogen chloride without isolating the donepezil base to form crude donepezil hydrochloride. The crude donepezil hydrochloride may be obtained in any known polymorph form, but preferably the polymorph form is form I, II or III. It is most desirable to prepare crude donepezil hydrochloride in the polymorph of form I. It is preferably to add crystalline donepezil hydrochloride form I as seeding material before precipitating the crude donepezil hydrochloride. The hydrogen chloride is gaseous hydrogen chloride or a solution of hydrogen chloride in solvent. The solvent is an organic solvent, water or a mixture thereof. Preferably the organic solvent is methanol, ethanol, isopropanol, ethyl acetate, diethyl ether, diisopropyl ether, 1,4-dioxane or a mixture thereof.

Donepezil hydrochloride in the form of polymorph I may be prepared by recrystallization of the crude donepezil hydrochloride from methanol.

Preferably the form I of donepezil hydrochloride is prepared by dissolving donepezil hydrochloride in 0-5% aqueous methanol, followed by addition of antisolvent. Preferably the antisolvent is diethyl ether or diisopropyl ether. It is preferable to add crystalline donepezil hydrochloride form I as seeding material in the process of the crystallization. The precipitate is filtered off and dried at 25-50° C. under reduced pressure to give purified donepezil hydrochloride in the form of polymorph I, with a LC purity of more than 99% and with a content of each individual impurity not exceeding 0.02%.

Donepezil hydrochloride form I hydrate may be prepared from donepezil hydrochloride form I with any water content and maturing the salt in a damp atmosphere. As a damp atmosphere, filtered air of the production area with 10-96% humidity may be used.

The prepared donepezil hydrochloride form I hydrate is characterized by 4 to 6% of water content and peaks expressed in degrees 2θ at approximately 9.8, 10.5, 12.6, 13.0, 13.6, 13.8, 14.8, 16.0, 16.8, 17.5, 19.8, 21.0, 21.2, 23.0 and 23.9 in a powder x-ray diffraction pattern.

The following abbreviations are used:
aq=aqueous;
Bn=benzyl;
DRIFTS=diffuse reflectance infrared;
DSC=differential scanning calorimetry;
FT-IR=Fourier-transform infrared;
HPLC=high performance liquid chromatography;
INN=international nonproprietary name;
IPA=isopropanol;
IR=infrared;
LC=liquid chromatography;
MTBE=tert-butyl methyl ether;
rt=room temperature;
TGA=thermograviometric analysis;
HCl=hydrogen chloride or hydrochloric acid;

The following example is illustrative, but not limiting, of the methods of the present invention. Other suitable modifications and adaptations of the variety of conditions and parameters normally encountered in chemical synthesis and which are obvious to those skilled in the art are within the spirit and scope of the invention.

Experimental Section:

All reagents and solvents were purchased from Aldrich Chemical Company unless specified otherwise and used without further purification.

All reactions were provided under argon or nitrogen atmosphere.

The LC purity was determined by separating a sample by high performance liquid chromatography (HPLC) under the following conditions, and calculating the area percentage thereof of each peak.

HPLC Conditions:
Column: Intersil® 5 μm ODS-2 150×4.6 mm
Mobile phase: MeCN/H₂O/70% aq HClO₄ 30:70:0.1 (v/v)
Flow rate: 0.7 mL/min
Column temperature: 36° C.
Detection: UV 230 nm
Sample concentration/Solvent: ˜0.5 mg per 1 mL of mobile phase diluent

Infrared (IR) absorbance spectra were obtained on a Nicolet Impact 410 FT-IR spectrophotometer using a neat liquid sample or dispersion of solid sample material in KBr or Nujol. Infrared (IR) reflectance spectra were obtained on a Nicolet Impact 410 FT-IR spectrophotometer equipped with Pike Technologies EasiDiff Diffuse Reflectance Accessory using a dispersion of solid sample material in KBr.

Powder x-ray diffraction patterns were obtained by methods known in the art using PANALYTICAL (Philips) X'Pert Pro MPD x-ray powder diffraction system (CuK_{α} radiation, PW3050/60 goniometer, PW3015/20 X'Celerator detector). The Bragg-Brentano scheme was used for beam focusing.

Melting points were determined in open capillary tubes with Buchi B-545 capillary melting point apparatus or with Mettler-Toledo FP-81 Melting point accessory with FP-900 processor and are uncorrected. The melting points generally depend upon level of purity of the samples.

Measurements of difference between the heat flows to a sample and a reference pan that are subject to the same temperature program (differential scanning calorimetry, DSC) were obtained on a Mettler-Toledo DSC 822e Differential Scanning Calorimeter.

Measurements of mass of a sample which is subject to a temperature program (thermogravimetric analysis, TGA) were obtained on a Mettler-Toledo TGA/SDTA 851e.

### EXAMPLE 1

Donepezil Hydrochloride Form I

A mixture of MeSO₃H (23.9 g, 249 mmol) and P₂O₅ (2.5 g, 18 mmol) was stirred at 70-80° C. until completely homogenized. N-Benzyl-2-(3,4-dimethoxybenzyl)-3-(4-piperidine)propionic acid (4.5 g, 11.3 mmol) and CH₂Cl₂ (18 mL) were added to the mixture at 15-30° C. The obtained mixture was refluxed for 3 h, cold to rt, treated with ice water and basified to pH 9-10 with 40% KOH. The aqueous layer was separated and extracted with CH₂Cl₂. The combined organic layer was dried over Na₂SO₄ and filtered. By repeated addition of MeOH and distillation under atmosphere pressure to a final pot temperature of 64-65° C. and a final volume of 15-18 mL, CH₂Cl₂ was displaced with MeOH to give a solution of crude donepezil base in MeOH. The solution was acidified with HCl. The obtained mixture was refluxed for 0.1-1 h, cooled to rt and seeded with crystalline donepezil hydrochloride. The mixture was cooled to 0-5° C., treated with i-Pr₂O (25-45 mL) and stirred for 0.1-1 h at 0-5° C. The precipitate was filtered off and dried in vacuo at 30-50° C. to give crude crystalline donepezil hydrochloride with 80-90% yield and more than 99% HPLC purity (by area). A mixture of the crude donepezil hydrochloride and methanol containing 0-5% of water (about 5 mL on 1 g of donepezil hydrochloride) was refluxed for 0.1-1 h, cooled to rt and seeded with donepezil hydrochloride which was characterized by peaks expressed in degrees 2□ at approximately 9.8, 10.5, 12.6, 13.0, 13.6, 13.8, 14.8, 16.0, 16.8, 17.5, 19.8, 21.0, 21.2, 23.0 and 23.9 in a powder x-ray diffraction pattern. The mixture was cooled to 0-5° C., treated with i-Pr₂O (6-10 mL on 1 g of the donepezil hydrochloride) and stirred for 0.1-1 h at 0-5° C. The precipitate was filtered off and dried in vacuo to give donepezil hydrochloride which was characterized by peaks expressed in degrees 2θ at approximately 9.8, 10.5, 12.6, 13.0, 13.6, 13.8, 14.8, 16.0, 16.8, 17.5, 19.8, 21.0, 21.2, 23.0 and 23.9 in a powder x-ray diffraction pattern, 0-5% of water content and a LC purity of more than 99.8%, with a content of each individual impurity not exceeding 0.02%. Then the donepezil hydrochloride was matured in a filtered laboratory atmosphere (about 40 to about 80% humidity) at 25-50° C. to give donepezil hydrochloride with 4-6% of water content and the same LC purity.

The x-ray powder diffraction pattern of the donepezil hydrochloride has characteristic peaks expressed in degrees 2θ at approximately 9.8, 10.5, 12.6, 13.0, 13.6, 13.8, 14.8, 16.0, 16.8, 17.5, 19.8, 21.0, 21.2, 23.0, 23.9, 29.0 and 29.4.

IR DRIFTS (KBr): 3587, 3365, 3253, 3072, 3002, 2937, 2856, 2534, 1684, 1643, 1604, 1592, 1500, 1477, 1457, 1452, 1438, 1431, 1409, 1368, 1315, 1266, 1250, 1230, 1217, 1191, 1175, 1157, 1120, 1103, 1067, 1038, 1011, 972, 950, 921, 896, 859, 835, 810, 798, 783, 758, 750, 700, 653, 603, 589 and 564 cm⁻¹.

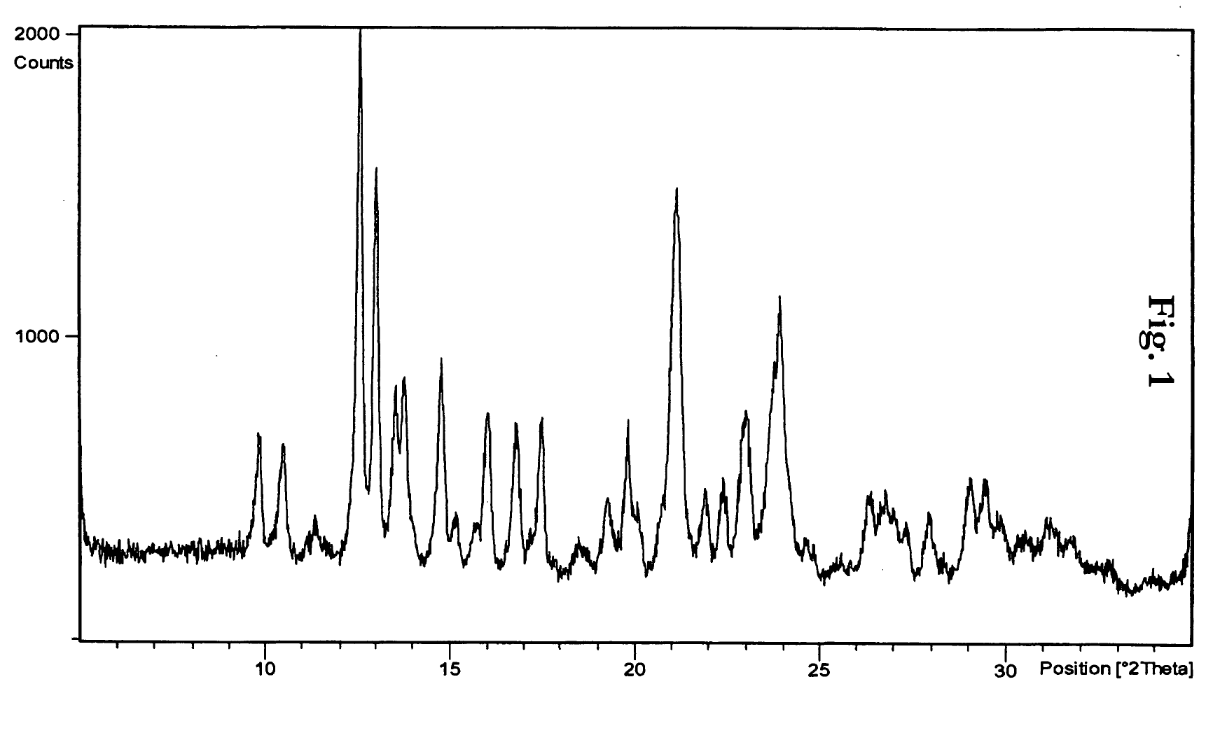

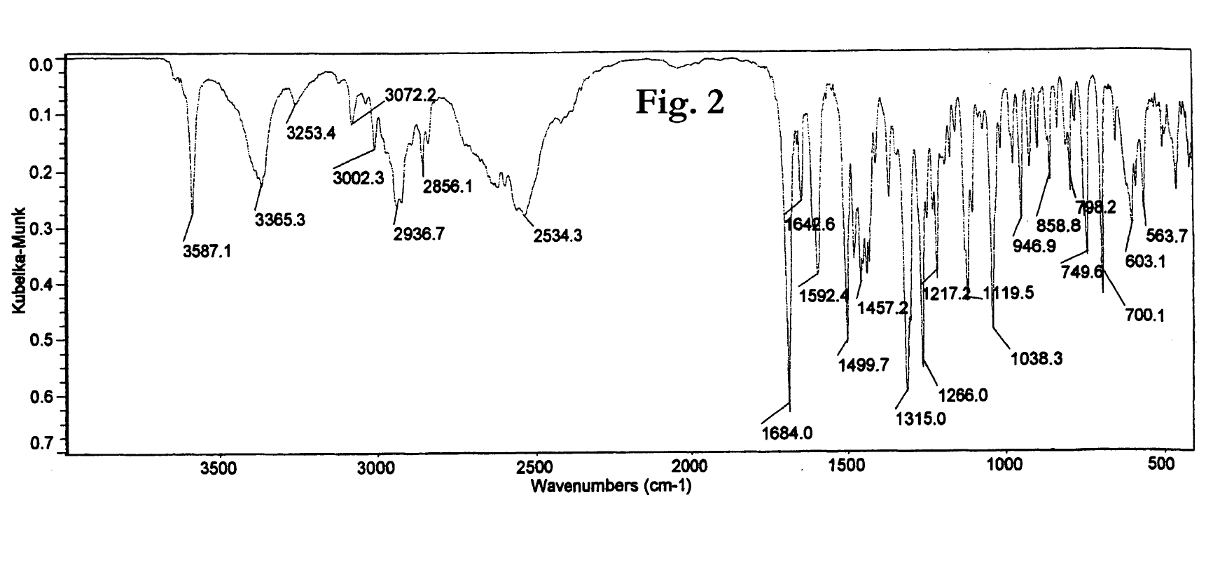

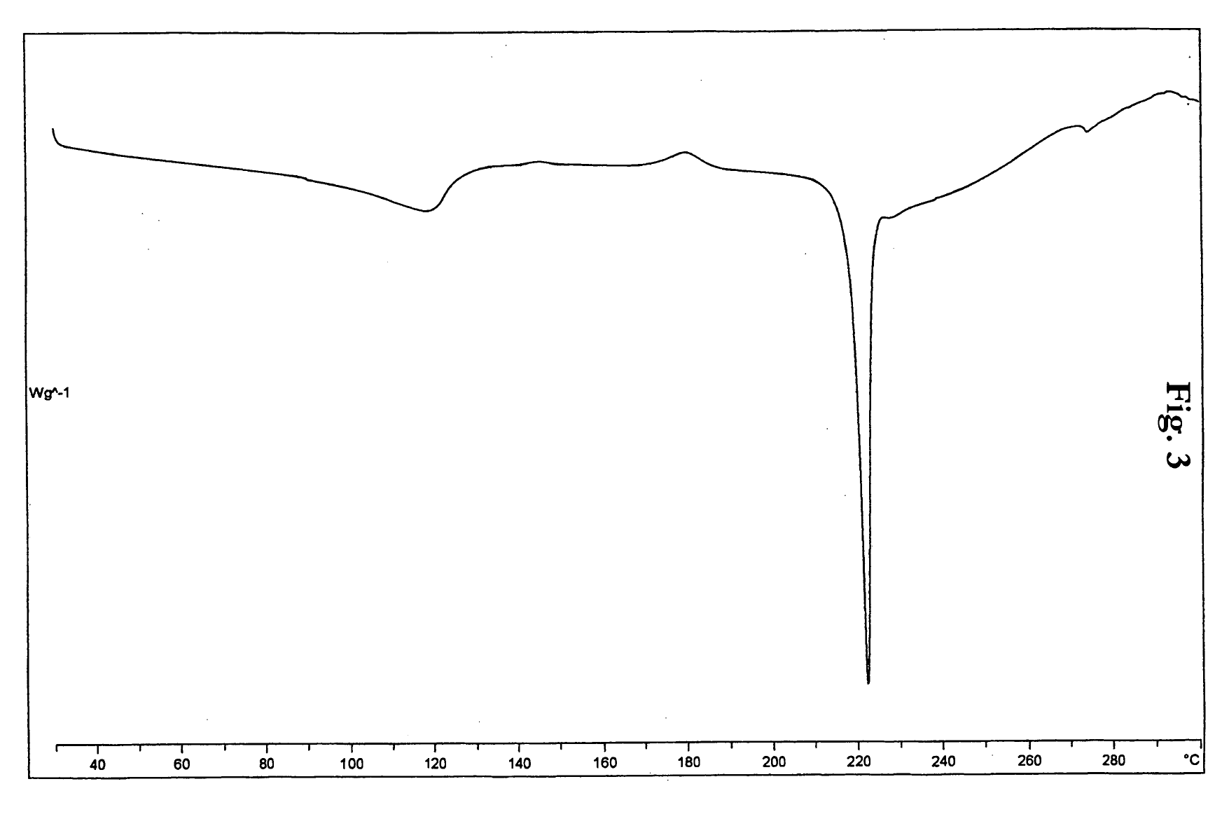

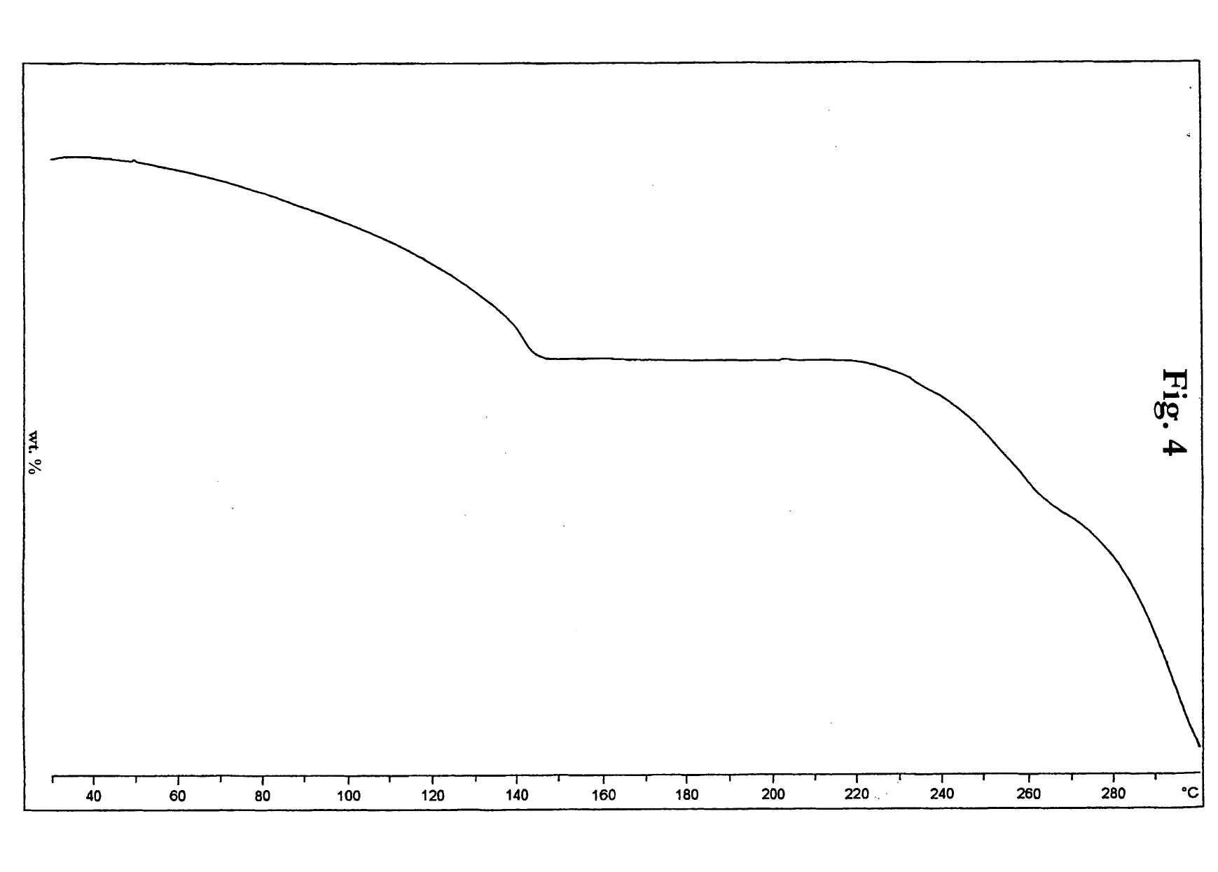

The obtained donepezil hydrochloride was characterized by powder x-ray diffractometry, IR DRIFTS (KBr) spectroscopy, DSC and TGA as set forth above and in FIGS. 1, 2, 3 and 4.

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

1. A process for the preparation of donepezil hydrochloride in the form of polymorph I, wherein the salt is characterized by peaks expressed in degrees 2θ at approximately 9.8, 10.5, 12.6, 13.0, 13.6, 13.8, 14.8, 16.0, 16.8, 17.5, 19.8, 21.0, 21.2, 23.0 and 23.9 in a powder x-ray diffraction pattern, 4 to 6% of water content, and a LC purity of more than 99%, with a content of each individual impurity not exceeding 0.02%, which process comprises:
carrying out an intramolecular cyclization of N-benzyl-2-(3,4-dimethoxybenzyl)-3-(4-piperidine)propionic acid or its salt to form donepezil base;
treating the donepezil base with HCl without isolating the donepezil base to form donepezil hydrochloride;
crystallizing the donepezil hydrochloride to give crystalline form I of donepezil hydrochloride; and
maturing the donepezil hydrochloride form I in a damp atmosphere to give donepezil hydrochloride of desired crystalline form, water content and LC purity.

2. The process of claim 1, wherein the intramolecular cyclization is performed in the presence of a protic acid, a Lewis acid, or a mixture thereof.

3. The process of claim 2, wherein said intramolecular cyclization is performed in the presence of a protic acid selected from the group consisting of trifluoromethanesulfonic acid, methanesulfonic acid, polyphosphoric acid, fluorosulfonic acid, chlorosulfonic acid, sulfuric acid, hydrogen fluoride, and hydrogen chloride.

4. The process of claim 2, wherein said intramolecular cyclization is performed in the presence of a Lewis acid selected from the group consisting of zinc chloride, zinc bromide, aluminum chloride, aluminum bromide, titanium chloride, boron fluoride, phosphorus pentoxide, phosphorus oxychloride, phosphorus pentachloride, phosphorus trichloride, thionyl chloride, and sulfuryl chloride.

5. The process of claim 1, wherein said intramolecular cyclization is carried out in the present of a solvent.

6. The process of claim 5, wherein said solvent is a halogenated solvent.

7. The process of claim 6, wherein said halogenated solvent is selected from the group consisting of dichloromethane, chloroform, dichloroethane, tetrachloroethane, chlorobenzene, and dichlorobenzene and mixtures thereof.

8. The process of claim 5, wherein said solvent is selected from the group consisting of nitromethane, nitroethane, nitrobenzene, and ether and mixtures thereof.

9. The process of claim 1, wherein the carboxylic group of N-benzyl-2-(3,4-dimethoxybenzyl)-3-(4-piperidine)propionic acid is derivatized to a halocarbonyl group prior to carrying out the intramolecular cyclization.

10. The process of claim 1, wherein the donepezil hydrochloride has a LC purity of more than 99.5%.

11. The process of claim 1, wherein the donepezil hydrochloride has a LC purity of more than 99.8%.

12. The process of claim 1, wherein the donepezil hydrochloride has a LC purity of more than 99.9%.

13. Donepezil hydrochloride in the form of polymorph I being specified by peaks expressed in degrees 20 at approximately 9.8, 10.5, 12.6, 13.0, 13.6, 13.8, 14.8, 16.0, 16.8, 17.5, 19.8, 21.0, 21.2, 23.0 and 23.9 in a powder x-ray diffraction pattern, 4 to 6% of water content, and a LC purity of more than 99.9%, with a content of each individual impurity not exceeding 0.02%, prepared by the process of claim 1.
